(19) United States
(12) Patent Application Publication
(10) Pub. No.: US 2022/0248949 A1
(43) Pub. Date: Aug. 11, 2022

(54) DEVICE AND METHODS FOR ASSESSING COLOR VISION

(71) Applicant: Health Research, Inc., Menands, NY (US)

(72) Inventors: James Norton, Albany, NY (US); Jonathan Carp, Albany, NY (US); Jonathan Wolpaw, Albany, NY (US)

(21) Appl. No.: 17/546,039

(22) Filed: Dec. 8, 2021

Related U.S. Application Data
(60) Provisional application No. 63/207,144, filed on Feb. 9, 2021.

(51) Int. Cl. A61B 3/06; A61B 3/00

(52) U.S. Cl. CPC A61B 3/066; A61B 3/0008

(57) ABSTRACT

The present disclosure relates to a method for assessing color vision. The method includes identifying one or more steady-state visual evoked potentials (SSVEPs) to identify metamers. The present disclosure further relates to devices for assessing color vision that use metamers identified via steady-state visual evoked potentials (SSVEPs). Also disclosed herein are methods of treating color vision deficiency, systems for identifying a response to one or more metameric stimuli, methods of individually modifying color vision, methods for assessing color vision using neural activity as a means to personalize visual displays, and methods for assessing light sensitive cells in the nervous system using flashing lights.

This application claims benefit of U.S. Provisional Patent Application Ser. No. 63/207,144, filed Feb. 9, 2021, which is hereby incorporated by reference in its entirety.

This invention was made with government support under 7P41EB018783 awarded by the National Institutes of Health and 5101CX001812 awarded by the Veterans Administration. The government has certain rights in the invention.

### FIELD

The present disclosure relates to devices and methods for assessing color vision.

### BACKGROUND

Accurate assessment of color vision is essential in many situations. Clinicians need it to detect and characterize the types and severities of the color vision deficits (CVDs) that affect >20% of the world population [1]; industry needs it to develop and validate color schemes for physical products and digital displays; and scientists need it to study the neural mechanisms of color vision and its variations across people.

The anomaloscope—which is based on color matching—is the current gold-standard method for assessing color vision due to its ability to identify the type and severity of CVDs [2]. With this method, the individual under study manually adjusts two light sources, one composed of a single wavelength and the other composed of two different wavelengths, until they appear to be the same color (i.e. are metamers; FIG. 1A). The light sources that different people perceive to be metamers vary due to differences in genetics, age, and sex (reviewed in [3]). By analyzing metamers, it is possible to assess how individuals see colors and to detect and characterize CVDs.

There are deficiencies in available methods and devices for accurate assessment of color vision. At present, color vision is evaluated mainly by behavioral methods, all of which require the attention and active participation of the subject. Moreover, current methods for assessing color vision require extensive training of the examiner and considerable time to administer.

The present disclosure is directed to overcoming these and other deficiencies in the art.

### SUMMARY

A first aspect relates to a method for assessing color vision. The method includes identifying one or more steady-state visual evoked potentials (SSVEPs) to identify metamers.

A second aspect relates to a device for assessing color vision. The device uses metamers identified via steady-state visual evoked potentials (SSVEPs).

A third aspect relates to a method of treating color vision deficiency. The method includes measuring one or more metamers identified via steady-state visual evoked potentials (SSVEPs), and administering a treatment for color vision deficiency.

A fourth aspect relates to a method of treating color vision deficiency. The method includes measuring a response to metameric stimuli identified by the device described herein, and administering a treatment for color vision deficiency.

A fifth aspect relates to a system for identifying a response to one or more metameric stimuli. The system includes providing one or more steady-state visual evoked potentials (SSVEP), and identifying one or more metamers.

A sixth aspect relates to a method of individually modifying color vision. The method includes utilizing feedback of brain activity elicited in response to metamers and colors that are close to being metamers identified by the methods described herein.

A seventh aspect relates to a method for assessing color vision. The method includes measuring neural activity using a human-computer interface or brain-computer interface.

An eighth aspect relates to a method for assessing color vision using neural activity as a means to personalize visual displays.

A ninth aspect relates to a method for assessing light sensitive cells in the nervous system using flashing lights.

Present methods for assessing color vision require the person's active participation. Here a brain-computer interface-based method is described for assessing color vision that does not require the person's participation. This method uses steady-state visual evoked potentials to identify metamers—two light sources that have different spectral distributions but appear to the person to be the same color. It is demonstrated that minimization of the visual evoked potential elicited by two flickering light sources identifies the metamer; this approach can distinguish people with color-vision deficits from those with normal color vision; and this metamer-identification process can be automated. This new method has numerous potential clinical, scientific, and industrial applications.

Here, a new way to identify metamers is presented. It is based on color matching, but—unlike existing methods—it does not require the active participation of the person being tested. Instead, it uses a noninvasive measure of brain activity (i.e. electroencephalography [EEG]) to quantify the brain's response to flickering lights (i.e. the steady-state visual evoked potential [SSVEP]).

When a visual stimulus alternates between two light sources at a set frequency, chromaticity and/or brightness differences between the two sources elicit brain activity (i.e. an SSVEP) at the same frequency as the alternation (see [4] for review, which is hereby incorporated by reference in its entirety; see also FIG. 4).

It was hypothesized that a stimulus alternating between metameric light sources will elicit little or no SSVEP. If this is correct, metamers should be identifiable as the pair of alternating light sources that minimize the SSVEP.

To test this hypothesis, a stimulator was designed that can produce metameric stimuli. It comprises three independently-controlled LEDs with wavelengths of 525 nm (green), 590 nm (amber), and 625 nm (red), respectively. These three LEDs can generate a monochromatic (amber) light source and a dichromatic (red and green) light source (FIG. 1A) that are metamers.

### BRIEF DESCRIPTION OF THE DRAWINGS

FIGS. 1A-1D show results of Experiment 1. FIG. 1A shows production of metameric stimuli. 525 nm (left) and 625 nm (middle-left) LEDs produce light that appears green and red, respectively. When appropriately combined (middle-right), these two LEDs form a dichromatic amber source that is a metamer of a monochromatic source from a 590 nm LED (right; also amber). FIG. 1B shows Experiment 1: Behavioral Session. Each of eight participants with normal color vision adjusts the dichromatic source from an initially randomized setting (red square) to a setting (cyan circle) that produces a light metameric with a monochromatic source of 600 D/A units luminance. FIG. 1C shows Experiment 1: SSVEP session (Exp. 1B). SSVEP size is measured by canonical correlation analysis as a function of the luminance of the amber monochromatic source. Values are baselined and then averaged across runs and participants. The vertical dashed line indicates the amber monochromatic luminance of 600 D/A units which the participants matched in Exp. 1A. The three insets show for one participant the EEG power spectra obtained at three different luminances of the monochromatic source. The SSVEP fundamental (10 Hz) and second harmonic (20 Hz) peaks nearly disappear at a luminance of 600, which is when the monochromatic and dichromatic sources are metamers. FIG. 1D shows spectrogram showing frequency-related changes in EEG activity for one participant as a function of the luminance of the amber monochromatic source. Data are the average of the three SSVEP runs for EEG channel Oz. As FIGS. 1C and 1D illustrate, the SSVEP peaks are minimal or absent when the dichromatic and monochromatic lights are metamers.

FIGS. 2A-2C show results of Experiments 2 and 3. FIG. 2A shows Experiment 2: Behavioral Session (Exp. 2A). Randomly initialized settings (red squares) and final settings (cyan circles) of the stimulator for seven participants with normal color vision. The cyan circles are the settings of the dichromatic source that the participants identified as being metamers to the monochromatic source, which was at a fixed luminance of 600 D/A units. FIG. 2B shows Experiment 2: SSVEP Session (Exp. 2B). Results for the (left) coarse-grid search and (right) fine-grid search. The data from the two grid searches are the average of all five runs from all seven participants. FIG. 2C shows Experiment 3. Coarse-grid search results for: (top) three representative participants with normal color vision; and (bottom) the three participants with a CVD. Each person with normal color vision shows an SSVEP minimum that is close to zero and is focused in both the green and red dimensions; those with CVDs do not do so.

FIG. 3 shows results of Experiment 4. Automated BCI-based metamer identification in three people with normal color vision. Each panel shows for one person the initial (black) and final (red) stimulator settings for each of three automated runs. In each person, the three runs begin from different locations; nevertheless, they end up very close to each other. The data are overlaid on the average SSVEP results of Experiment 2 (i.e. Exp. 2B). As expected for these three people with normal color vision, the final locations reached by the automated search are at or very close to the average SSVEP minimum for people with normal color vision.

FIG. 4 shows steady-state visual evoked potentials (SSVEPs). SSVEPs are elicited by alternating stimuli that differ in chromaticity, brightness, or both. Left and Center: A stimulus (left) that alternates between a red light (red dashed line) and a green light (green solid line) at 10 Hz elicits brain activity (i.e., an SSVEP) at the fundamental and harmonic frequencies of the stimulation (middle). Right and Center: A light source that alternates between two amber lights (right) of different luminance settings (perceived to be different brightnesses) also elicits an SSVEP at the fundamental and harmonic frequencies of the stimulation (middle).

FIGS. 5A-5B show stimulator and BCI system diagrams. FIG. 5A shows stimulator diagrams as viewed from the (top left) top and (right) side. (bottom left) Diagram of the LZ4 emitter (based on schematics from the data sheet). Note that the emitter has four dies, each die is referred to as the red, green, blue, and amber LEDs respectively. FIG. 5B shows BCI System Diagram—The participant views the stimulator from ˜30 cm. The flashing stimulus elicits changes in EEG activity, which are transmitted to an amplifier. The amplifier sends these signals via USB to a computer for analysis. Adjustment of the stimulator starts with a command from the computer to the Teensy microcontroller via USB. The computer command causes the Teensy microcontroller to change the duty cycle (reported in digital to analog (D/A) units) of an analog output pin using pulse width modulation (PWM). This change in duty cycle from the analog output pin is then transmitted to the constant current controller, which subsequently changes the current to the LED. Increasing the current increases luminance, decreasing the current decreases luminance.

FIGS. 6A-6C show behavioral identification of metamers. FIG. 6A shows behavioral adjustment procedure. First the digital-to-analog (D/A) settings of the dichromatic source are randomly initialized. Then, the individual LEDs are adjusted iteratively. This process ends when three stopping criteria are met: (1) Single-LED calibration and dual-LED calibration are both completed; (2) In these calibrations, the red and green D/A settings change by <10 D/A units; and (3) The participant says that the monochromatic source and dichromatic source are the same color. FIG. 6B shows an example of a single-LED calibration, which mostly adjusts the chromaticity of the dichromatic source. The participant compares the monochromatic source to two dichromatic sources in a two-interval forced discrimination task (2IFD) and chooses the one that is closer in color to the monochromatic source. Based on the result, the 2IFD is repeated with the chosen alternative and a new alternative until the stopping criteria are met. This process is completed for the red LED and then for the green LED. FIG. 6C shows an example of dual-LED calibration, which mostly adjusts the brightness of the dichromatic source. The red and green LEDs are adjusted simultaneously with heterochromatic flicker photometry (HFP), in which the stimulus flickers at 25 Hz between the monochromatic source and a dichromatic source. In a 2IFD, the participant decides which of two alternative dichromatic sources causes less flicker when it alternates with the monochromatic source (and thus differs less in brightness). Based on the result, the 2IFD is repeated with the chosen alternative and a new alternative until the stopping criteria are met.

FIG. 7 shows Experiment One: SSVEP Session. These three panels illustrate the process that produced the three insets of FIG. 1C. As the luminance of the monochromatic source is gradually raised (in 20-D/A unit steps) from 0 to 1020 D/A units, the stimulator alternates between the monochromatic source and the dichromatic source (which is at the red/green luminance settings defined in the Behavioral Session of Experiment One). When the monochromatic source luminance is low (left) or when it is high (right), the SSVEP is large. When it is 600 D/A units, the SSVEP is minimal.

FIG. 8 shows Experiment One: SSVEP Session. Using a Google Pixel 4A (Lux Light Meter app), the meter was fixed ˜3.5 inches from the stimulator in a darkened room (i.e., 0 lux) and recorded a measurement of illumination for each setting used in Experiment One (i.e., 0 to 1020 in steps of 20) in the order they were presented during the experiments. The results show a linear increase in illumination as a function of trial number. Note that during the test, the meter was placed closer to the stimulator than the participant was during the experiments. This was done because it was found that the readings from the meter were more reliable at ˜3.5 inches than ˜12 inches. The red dot is the illuminance of the dichromatic source that the participant identified to be a metamer of the monochromatic source at a setting of 600 D/A units. Also note the larger difference in illuminance between low settings of the amber light source and high settings of the amber light source, randomizing the order of the trial may have introduced large changes in pupil size could have contributed significant noise to the experiments.

FIG. 9 shows topographical maps of data from Experiment One. SSVEP power is plotted as a function of electrode location for the (a) fundamental frequency (i.e., 10 Hz) and (b) second harmonic frequency (i.e., 20 Hz). Power spectra for each channel were calculated in MATLAB using the pwelch command (512 sample window size, 384 sample overlap, zero-padded to 2048 samples). Data from multiple subjects and runs were averaged and plotted using EEGLab's topoplot function (Delorme and Makeig (2004)). The three column represent monochromatic (i.e., amber) light source settings of 0, 600, and 1020 D/A units (from left to right).

FIGS. 10A-10C show that grid searches are a method for determining optimal values of hyperparameters. In Experiment Two, two grid searches were performed, a coarse grid and a fine grid search. FIG. 10A shows that the first step in each grid search was data collection. In FIG. 10B, following data collection, the data was analyzed by extracting each of the trials from each of the five runs. The maximum canonical correlation for each trial from each run was then extracted. Finally, the canonical correlations for each trial were averaged across runs. In FIG. 10C, the data was then visualized by mapping the average canonical correlations to the appropriate locations on the grid. Grids from different subjects were averaged to produce FIG. 2B; grids for individuals with CVDs were visualized in FIG. 2C.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### DETAILED DESCRIPTION

A first aspect relates to a method for assessing color vision. The method includes identifying one or more steady-state visual evoked potentials (SSVEPs) to identify metamers.

It is to be appreciated that certain aspects, modes, embodiments, variations, and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this disclosure belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±1 or ±10%, or any point therein, and remain within the scope of the disclosed embodiments.

Where a range of values is described, it should be understood that intervening values, unless the context clearly dictates otherwise, between the upper and lower limit of that range and any other stated or intervening value in other stated ranges, may be used in the embodiments described herein.

As used herein, the terms “subject”, “individual”, or “patient,” are used interchangeably, and mean any animal, including mammals, such as mice, rats, other rodents, rabbits, dogs, cats, swine, cattle, sheep, horses, or primates, such as humans.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

Steady-state visual evoked potentials (SSVEPs) as described herein include electroencephalogram (EEG) measures of the signals from cortical brain regions that respond in synchrony with a flickering visual stimulus, the signals represent brain responses that have reached a steady-state relationship with visual stimulus. The present disclosure includes methods, systems, and devices that acquire, process, and/or utilize Steady-state visual evoked potentials (SSVEPs) for monitoring, tracking, and/or diagnosing various paradigms in color vision efficiency and/or deficiency, particularly for SSVEPs generated by optical stimuli.

Metamers as described herein include two light sources that have different spectral distributions but appear to a subject (e.g., a person) to be the same color. When four or more non-coincident color primaries are used in a display, commonly called a “multiprimary” display in the art, there are often multiple combinations of values for the primaries that may give the same color value. That is to say, for a given hue, saturation, and brightness, there may be more than one set of intensity values of the four or more primaries that may give the same color impression to a human viewer. Each such possible intensity value set may be referred to as a “metamer” for that color. Thus, a metamer is a combination (or a set) of at least two groups of colored sub-pixels such that there exists signals that, when applied to each such group, yields a desired color that is perceived by the subject. Such a signal may vary from group of sub-pixels to group, in order to produce the same or substantially similar perceived color. Because of this, a degree of freedom exists to adjust relative values of the primaries for some effect.

In one embodiment, a brain computer interface (BCI) is used to identify metamers. The brain computer interface may include any device or measure suitable to capture information on brain structure and function. For example, the brain computer interface may include Magnetic Resonance Imaging (MRI), functional Magnetic Resonance Imaging (fMRI), magnetoencephalography (MEG), electroencephalogram (EEG), or any combination thereof. In one embodiment, the BCI is an EEG measurement.

In some implementations, for example, the methods, devices, and systems described herein include using SSVEP and brain-computer interfaces (BCIs) to bridge the human brain with computers or external devices. By detecting the SSVEP frequencies from a non-invasively recorded EEG, the users of SSVEP-based brain-computer interface can interact with or control external devices and/or environments through gazing at distinct frequency-coded targets.

In one embodiment, the metamers are in response to a metameric stimuli comprising a light source. A light source may, in one embodiment, be an LED light source, or alternatively, any suitable light source to produce a metameric stimuli. The light source may produce any suitable wavelength to produce a metameric stimuli. For example, the light source may produce a wavelength of about 525 nm (green), about 590 nm (amber), about 625 nm (red), or any combination thereof. In one embodiment, the light source comprises a wavelength between 400 nm and 700 nm. These three LEDs may, in one embodiment, generate a monochromatic (amber) light source and a dichromatic (red and green) light source that are metamers. In one embodiment, the metameric stimuli comprise a monochromatic light source or a dichromatic light source. In one embodiment, the metameric stimuli is alternating. In one embodiment, the metameric stimuli comprise at least two different stimuli, wherein said stimuli differ in color, hue, luminance, saturation, or any combination thereof.

In one embodiment, the method further comprises providing a subject having or suspected of having a color vision deficiency.

Color vision deficiency as described herein includes obstacles in recognizing colors. For example, subjects with red-green color blindness cannot distinguish between red and green. Color vision deficiency is very common and causes many difficulties in life for patients. Color vision deficiency is caused by problems occurring to the development cone cells, and the root cause is the deletion of genes on the chromosomes. It is a genetic disease which cannot be treated currently, and thus it is difficult to completely restore the level of a person with color vision deficiency to that of a normal person. The types of color vision deficiency may include anomalous trichromacy, dichromacy and monochromacy. People with said color vision deficiency may encounter difficulties in distinguishing colors in the images or any real time visual content. Color vision deficiency type or status may be indicated as normal, completely color-blind, or bichromatic color blindness.

In one embodiment, the method further comprises comparing presence of metamers and/or a response to metameric stimuli in said subject having or suspected of having a color vision deficiency to a control response to metameric stimuli.

In one embodiment, when the metamers and/or response to metameric stimuli is within a specific range, the metamers and/or response indicates that said subject has or is likely to have a color vision deficiency.

A second aspect relates to a device for assessing color vision. The device uses metamers identified via steady-state visual evoked potentials (SSVEPs).

This aspect may be in accordance with the previously described aspect.

In one embodiment, the device further includes a brain computer interface (BCI) to automatically identify metamers.

The brain computer interface may include any device or measure suitable to capture information on brain structure and function. For example, the brain computer interface may include Magnetic Resonance Imaging (MRI), functional Magnetic Resonance Imaging (fMRI), magnetoencephalography (MEG), electroencephalogram (EEG), or any combination thereof. In one embodiment, the BCI is an EEG measurement.

Methods, devices and systems of the disclosed technology can implement wireless SSVEP data acquisition and processing. Methods, devices and systems described herein may include a noninvasive platform for continuously monitoring high temporal resolution brain dynamics without requiring conductive gels applied to the scalp. For example, microelectromechanical system EEG sensors, low-power signal acquisition, amplification and digitization, wireless telemetry, and real-time processing may be used. In addition, the methods, devices, and systems described herein may include analytical techniques, such as independent component analysis, which can improve detectability of SSVEP signals.

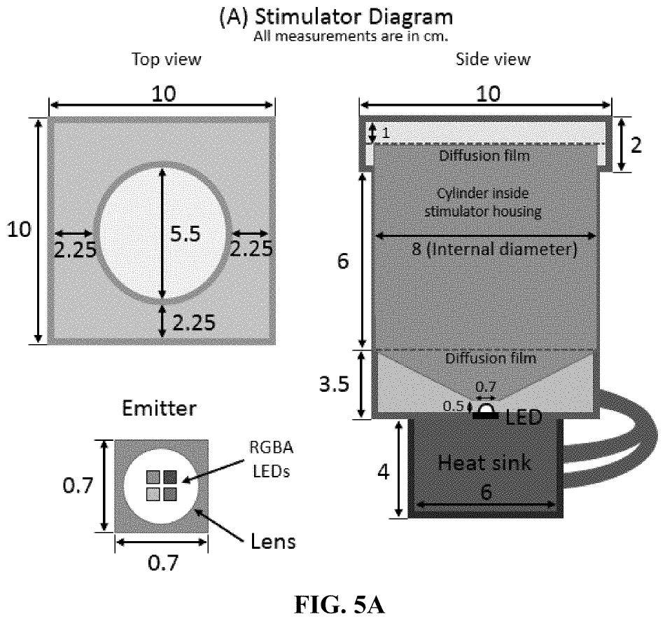

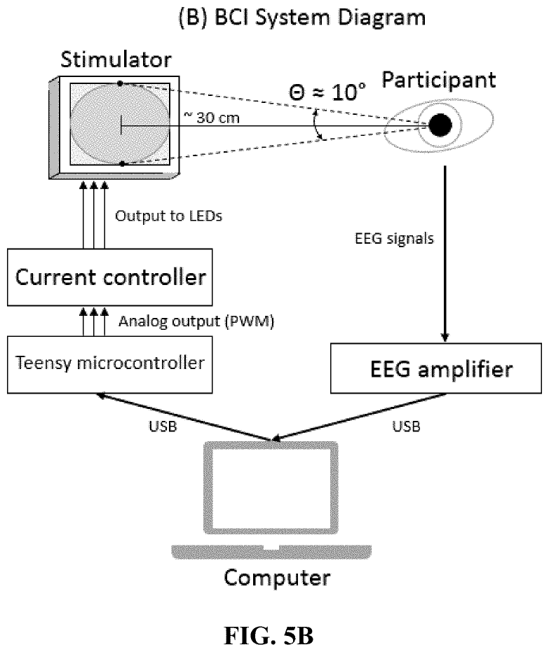

FIGS. 5A-5B show stimulator and BCI system diagrams in accordance with the device, methods, and systems described herein. FIG. 5A shows stimulator diagram that can produce metameric stimuli. The emitter, in one embodiment, has four dies, each die is referred to as the red, green, blue, and amber LEDs respectively. For example, it may include three independently-controlled LEDs with wavelengths of 525 nm (green), 590 nm (amber), and 625 nm (red), respectively. These three LEDs can, in one embodiment, generate a monochromatic (amber) light source and a dichromatic (red and green) light source that are metamers. FIG. 5B shows a BCI System Diagram in accordance with the device, methods, and systems described herein. The subject or participant may, in one embodiment, view the stimulator from about 30 cm. The flashing stimulus may, in one embodiment, elicit changes in EEG activity, which may be transmitted to an amplifier. The amplifier may, in one embodiment, send these signals via USB to a computer for analysis. Adjustment of the stimulator may, for example, start with a command from the computer to the Teensy microcontroller via USB. The computer command may, in one embodiment, cause the Teensy microcontroller to change the duty cycle (reported in digital to analog (D/A) units) of an analog output pin using pulse width modulation (PWM). This change in duty cycle from the analog output pin may, in one embodiment, then be transmitted to the constant current controller, which may subsequently change the current to the LED. Increasing the current may increase luminance, decreasing the current may decrease luminance.

In one embodiment, the device includes providing alternating metameric stimuli. In one embodiment, the metameric stimuli comprise at least two different stimuli, wherein said metameric stimuli differ in color, hue, luminance, saturation, or any combination thereof.

A third aspect relates to a method of treating color vision deficiency. The method includes measuring one or more metamers identified via steady-state visual evoked potentials (SSVEPs), and administering a treatment for color vision deficiency.

This aspect may be in accordance with the previously described aspects.

In one embodiment, the method further includes providing alternating metameric stimuli in accordance with the previously described aspects. In one embodiment, the metameric stimuli comprise at least two different stimuli, wherein said metameric stimuli differ in color, hue, luminance, saturation, or any combination thereof.

As used herein, the term “effective amount” includes an amount of a compound or pharmaceutical agent that will elicit the biological or medical response of a cell, tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of the compound of the present disclosure (e.g., metamers), as well as salts, solvates, and physiological functional derivatives thereof, may be administered as the raw chemical. Additionally, the active ingredient may be presented as a pharmaceutical composition.

For purposes of this and other aspects of the disclosure, the target “subject” encompasses any vertebrate, such as an animal, preferably a mammal, more preferably a human. In the context of administering a composition of the disclosure for purposes of assessing color vision in a subject comprising in a subject, the target subject encompasses any subject that is a target for color vision assessing. Subjects may include infants, juveniles, adults, or elderly adults. In one embodiment, the subject is an infant, a juvenile, or an adult. In one embodiment, the method is performed in a subject having a preexisting condition or, alternatively, may be performed in a subject having no preexisting condition. The method may also be performed on a subject who has been previously treated for a color vision deficits.

As used herein, the phrase “therapeutically effective amount” means an amount that elicits the biological or medicinal response that is being sought in a tissue, system, animal, individual, or human by a researcher, veterinarian, medical doctor, or other clinician. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment.

One goal of treatment is the amelioration, either partial or complete, either temporary or permanent, of patient symptoms. Any amelioration is considered successful treatment. This is especially true as amelioration of some magnitude may allow reduction of other medical or surgical treatment which may be more toxic or invasive to the patient. The treatment as described herein may further be used to train color vision in healthy individuals. The treatment described herein may be conducted non-invasively or invasively.

As used herein a sample may include any sample obtained from a living system or subject, including, for example, blood, serum, and/or tissue. In one embodiment, a sample is obtained through sampling by minimally invasive or non-invasive approaches (for example, by eye excretion, urine collection, stool collection, blood drawing, needle aspiration, and other procedures involving minimal risk, discomfort, or effort). Alternatively, samples may be gaseous (for example, breath that has been exhaled) or liquid fluid. Liquid samples may include, for example, eye secretion, urine, blood, serum, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, nasal excretions, and other liquids. Samples may also include a clinical sample such as serum, plasma, other biological fluid, or tissue samples, and also includes cells in culture, cell supernatants, and cell lysates. In one embodiment, the sample is selected from the group consisting of whole blood, serum, urine, and nasal excretion. Samples may be in vivo or ex vivo.

A treatment for color vision deficiency may include any method, device, or system that improves and/or treats a color vision deficiency. For example, a treatment may include SVEP-based operant conditioning protocol as described herein to improve color vision in people with color vision deficiency. A treatment may further include any additional treatment known to those skilled in the art for improving color vision deficiency, including, for example, wearing a pair of smart glasses, which, may include a device as described herein.

A fourth aspect relates to a method of treating color vision deficiency. The method includes measuring a response to metameric stimuli identified by the device described herein, and administering a treatment for color vision deficiency.

This aspect may be in accordance with the previously described aspects.

A fifth aspect relates to a system for identifying a response to one or more metameric stimuli. The system includes providing one or more steady-state visual evoked potentials (SSVEP), and identifying one or more metamers.

This aspect may be in accordance with the previously described aspects.

In one embodiment, the system is used to visualize information processing in the cortex of a subject. In one embodiment, a brain computer interface (BCI) is used to identify said response to one or more metameric stimuli in accordance with the previously described aspects.

In one embodiment, the one or more metameric stimuli comprise a light source in accordance with the previously described aspects. In one embodiment, the light source comprises a wavelength between 400 nm and 700 nm. In one embodiment, the metameric stimuli comprise a monochromatic light source or a dichromatic light source. In one embodiment, the subject has or is suspected of having a color vision deficiency.

In one embodiment, the metamers and/or response to metameric stimuli in said subject having or suspected of having a color vision deficiency is compared to a control metamer and/or response to metameric stimuli. In one embodiment, when said metamer and/or response to metameric stimuli in said subject having or suspected of having a color vision deficiency is within a specific range, said response indicates that said subject has or is likely to have a color vision deficiency.

In one embodiment, the system provides real-time analysis of brain activity. The system described herein may, in one embodiment, elicit and analyze a subject's SSVEP in real-time while iteratively adjusting the luminances of the green and red LEDs that produce the dichromatic source until the SSVEP is minimized, thereby identifying a metamer to the monochromatic amber source (the luminance of which is fixed throughout). This process of identifying the specific settings of the dichromatic source that are metameric to the monochromatic source is a two-dimensional optimization problem, which may be solved using gradient descent based on finite differences.

In one embodiment, a relative color of two light sources is adjusted until one or more metamers are identified.

A sixth aspect relates to a method of individually modifying color vision. The method includes utilizing feedback of brain activity elicited in response to metamers and colors that are close to being metamers identified by the methods described herein.

This aspect may be in accordance with the previously described aspects.

A seventh aspect relates to a method for assessing color vision. The method includes measuring neural activity using a human-computer interface or brain-computer interface.

This aspect may be in accordance with the previously described aspects.

In one embodiment, the measuring uses neural responses to flashing lights.

In one embodiment, the measuring is based on a neural imaging system and a flashing stimulator.

An eighth aspect relates to a method for assessing color vision using neural activity as a means to personalize visual displays.

This aspect may be in accordance with the previously described aspects.

A ninth aspect relates to a method for assessing light sensitive cells in the nervous system using flashing lights.

This aspect may be in accordance with the previously described aspects.

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense.

The present disclosure may be further illustrated by reference to the following examples. The examples are intended to illustrate, but by no means are intended to limit, the scope of the present disclosure as set forth in the appended claims.

### EXAMPLES

The following examples are intended to illustrate, but by no means are intended to limit, the scope of the present disclosure as set forth in the appended claims.

### Example 1—Materials and Methods

All experiments were approved by the institutional review board of the New York State Department of Health's Wadsworth Center.

Participants—Nineteen people were studied (five females and fourteen males, 20-72 years of age). Nine people completed Experiment One; nine people completed Experiment Two; three people completed Experiment Three; and three people completed Experiment Four. One person completed both Experiment One and Experiment Two (E1 (i.e. excluded)=S205), one person completed both Experiment Two and Experiment Four S201=S401), and two people completed experiments one, two, and four (S107=S206=S402 and S105=S207=S403). The data from one participant was excluded from Experiment One (E1) due to a technical issue. Data from two participants were excluded from Experiment Two because they did not generate a measurable SSVEP. The color vision of each of the participants was screened using the first 25 plates of the 38 plate Ishihara pseudoisochromatic test [26] and/or the Farnsworth D-15 test [27]. One of the participants without CVDs completed their screening online and two others self-reported having no CVD.

EEG recording—EEG was recorded using a 16-channel g.USB (g.tec Medical Engineering GmbH, Austria) B-series amplifier. EEG electrodes were located in a mesh cap (Electro-Cap International, Inc. Eaton, Ohio) at the following 10-10 international system locations: F3, Fz, F4, T7, C3, CZ, C4, T8, CP3, CP4, P3, Pz, P4, PO7, PO8, and Oz [28]. During the recordings, the EEG data were referenced to an electrode over the right mastoid and a ground electrode was placed on the left mastoid. Conductive gel was used to reduce to impedance to <40 kΩ. All data were acquired using BCI2000 [29] at a sampling rate of 256 Hz. No bandpass or notch filters were used during acquisition.

Stimulation system—A custom-built stimulation system, consisting of a stimulator, microcontroller, and software interface was used to elicit SSVEPs. Diagrams of the stimulator (including the emitter) and BCI system are shown in FIGS. 5A-5B. The stimulator consisted of a four-die (red [˜625 nm; full width at half maximum (FWHM)=20 nm], amber [˜590 nm; FWHM=20 nm], green [˜525 nm; FWHM=35 nm], and blue [˜465 nm]) LED emitter (LZA4-00MA00; LED Engin, Inc. San Jose, Calif.), heat sink, constant current LED driver (DD313, Silicon Touch Technology, Inc. Hsin-Chu, Taiwan), and 3D printed housing. In this paper, each die is referred to as an LED.

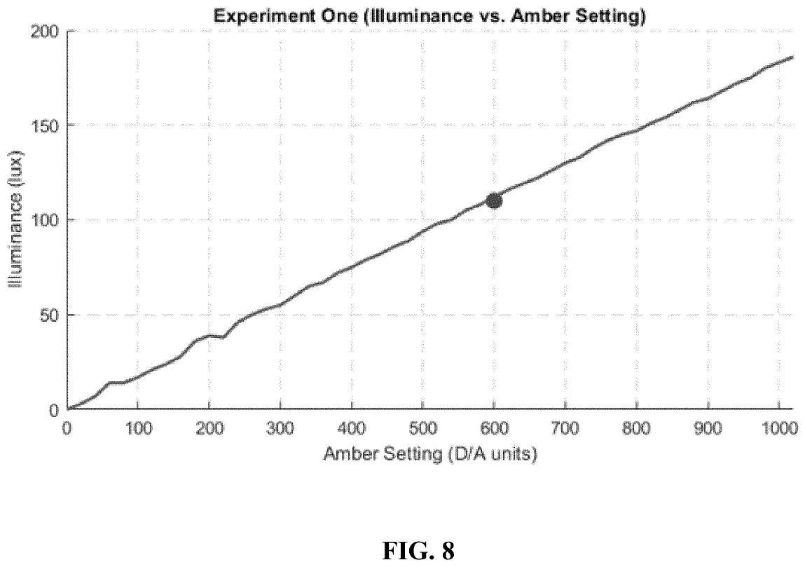

The stimulator was controlled by a Teensy microcontroller (PJRC, Sherwood, Oreg.) running Arduino (Arduino LLC, Somerville, Mass.). A software interface was developed with MATLAB (The Mathworks Inc. Natick, Mass.) to allow the experimenters to digitally adjust the luminance of each LED using pulse-width modulation (PWM; see FIG. 8).

PWM adjusts the proportion of the time that the voltage input to the LED is high (i.e. on). In the design, PWM was adjusted with 10-bits of precision (i.e. there were 1024 possible luminance settings for each LED). These potential settings are referred to as D/A units. A setting of 0 denoted that the input to the LED was high 0% of the time and a setting of 1023 represented that the input voltage to the LED was high 100% of the time.

Digital control of the luminance of the LEDs starts at the computer (FIGS. 5A-5B and 8). A command to increase the luminance of one of the LEDs is sent from the computer to the Teensy microcontroller via USB. The computer command causes the Teensy microcontroller to increase the duty cycle (in D/A units) of an analog output pin using PWM. PWM adjusts the proportion of the time that the voltage output of an analog output pin is high. In the design, PWM was adjusted with 10-bits of precision (i.e. there were 1024 possible settings; measured in D/A units from 0 to 1023). This increased duty cycle from the analog output pin is then transmitted to the constant current controller. The constant current controller adjusts the current to the LED based on PWM (i.e. higher values of PWM output higher current). Given the fixed position of the LED and diffusers, the projected geometry of the light passing through the stimulator is also fixed. Thus, increasing current increases luminance as perceived by the participant.

Procedure—All experiments were conducted at the David Axelrod Institute, Wadsworth Center, Albany, N.Y. in a room with consistent ambient lighting (˜300 lux).

After completing the informed consent process, participants were asked to sit in a comfortable desk chair for the duration of the study. A chin rest was used during all of the sessions to stabilize the position of each participant relative to the stimulator. Participants were about one foot from the stimulator; it subtended a visual angle of ˜10°.

Experiment one—Experiment One compared behaviorally-identified metamers with those identified using SSVEPs.

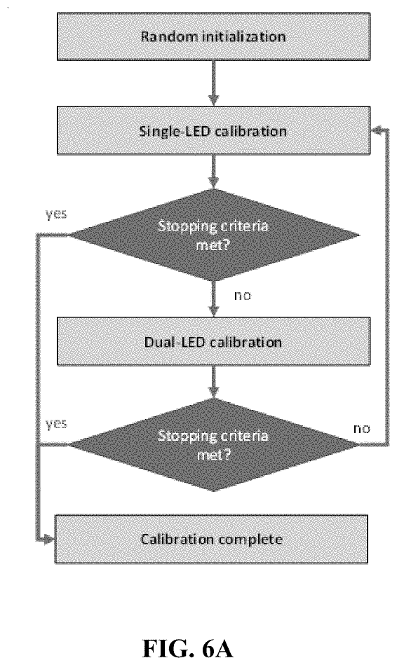

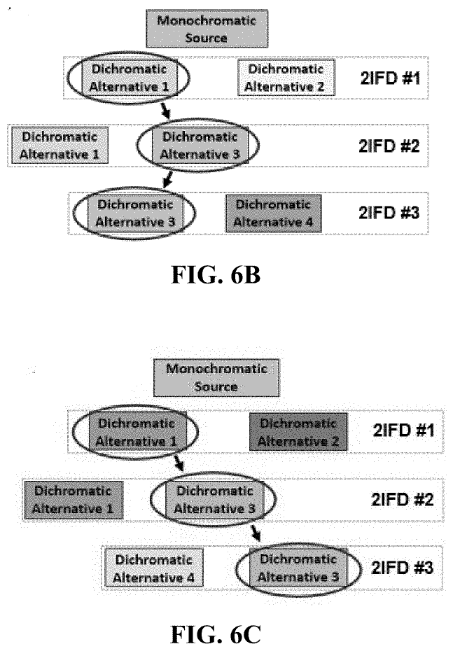

Behavioral session—To account for individual differences in color [3, 30], a behavioral session (Exp. 1A) was used to find a combination of red and green lights that had the same color as the amber light at a predetermined luminance setting (600 D/A units). This behavioral session was completed in multiple phases (FIGS. 6A-6C): initialization; iterative adjustment process (i.e. single- and dual-LED calibration); and stopping.

Initialization—During initialization, the amber light (monochromatic source) was always set to have an D/A setting of 600. The red and green lights (dichromatic source), however, were randomly set to have D/A settings between 0 and 255. After initialization, the participant was asked for guidance on how to adjust the stimulator (e.g. is the test source too green?). Based on this guidance, the experimenter could choose one of two different actions. For example, if the participant indicated that the test source was too green, the experimenter could either (1) decrease the luminance of the green LED or (2) increase the luminance of the red LED. The choice of action was left to the discretion of the experimenter.

Single-LED calibration—A two-interval discrimination task (2IDT) was used to adjust the dichromatic source to have the same color as the monochromatic source (FIG. 6B). To do this, the participants were asked to judge which of two test sources was closer in color to the monochromatic source. The two alternatives consisted of different combinations of light from the red and green lights. The luminances of the red and green lights were adjusted separately (red first and then green). As an example of adjusting the red light, two alternative dichromatic light combinations are presented to the participant. In Alternative 1, the red and green lights have D/A settings of 250 and 50 respectively, while in Alternative 2, the red and green lights have D/A settings of 200 and 50 respectively. During this presentation, the stimulator switches between the monochromatic source and one of the two alternative dichromatic sources at a rate of 1 Hz. Each presentation lasts for 3 s. The participant is then asked which of the two alternatives is more similar in color to the monochromatic source. If the participant is unsure, they are permitted to see each alternative again. Based on the participant's answer, the luminance of the red light is then increased or decreased. The relative size of the luminance change—known as the increment—was determined a priori and started at 50 D/A units.

Continuing this example, if the participant chooses Alternative 1, the new 2IDT would be between Alternative 1's current red and green D/A settings and a new alternative (i.e. Alternative 3) with D/A settings of 300 and 50. If the participant chooses Alternative 3, the process continues. If the participant chooses Alternative 1 again, the size of the increment is reduced (from 50 to 25 after the choice is repeated once, and then from 25 to 10 D/A units after the choice is repeated a second time). The process is then resumed with a new alternative (i.e. Alternative 4, with settings of 275 and 50]). Calibration of the red light is stopped when the increment equaled 10 and the same alternative is chosen twice in a row. This process is then repeated for the green light.

Dual-LED calibration—Dual-LED calibration (where both the red and green LEDs were adjusted simultaneously) was based on heterochromatic flicker photometry (HFP) [31-33]. Originally described by Walsh [31], HFP is a method for comparing the brightness of two light sources [32]. When flickered sufficiently fast (i.e. above the critical flicker fusion rate [31]) light sources of equal brightness have minimal perceived flicker. In the present disclosure, participants were asked to minimize the flicker between two light sources at a single location using a 2IDT (FIG. 6C). Each of the two alternatives consisted of the monochromatic source and a dichromatic source. By alternating between these two light sources (at 25 Hz) [34, 35], a flickering stimulus was generated. As an example, Alternative 1 consisted of the monochromatic source and a dichromatic source with red and green D/A settings of 200 and 50. Alternative 2 consisted of the monochromatic source and a dichromatic source with red and green D/A settings of 100 and 25. These two alternatives were then presented to the participant for 3 s each. The participant was then asked which of the two alternatives flickered less. If the participant was unsure, they were permitted to see each alternative again. Based on the participant's answer, the luminance of the red and green lights was then increased or decreased. To primarily adjust the brightness of the test source, the luminance of both the red and green lights were increased or decreased simultaneously. As in the monochromatic calibration, the amount that the lights were adjusted after each decision was defined as the increment. The increment during dichromatic calibration started at 100 D/A units. Continuing the example, if the participant chose Alternative 2, the new 2IDT would be Alternative 2 and a new alternative (i.e. Alternative 3) with increased D/A settings (300 and 75). If the participant chose Alternative 3, the process continued. If the participant chose Alternative 2 again, the size of the increment was reduced (from 100 to 50 after the choice is repeated once, then from 25 to 10 D/A units after the choice is repeated a second time) and a new alternative was presented. Dichromatic calibration stopped when the increment equaled 10 and the same alternative was chosen twice in a row.

Stopping criteria—Both the single-LED and dual-LED calibrations were each repeated until three stopping criteria were met. First, both the single-LED and dual-LED calibrations were completed at least once. Second, the total difference between the current red and green light D/A settings were no more than ten units different from the red or green light D/A settings from the previous single-LED or dual-LED calibration. Third, the participant confirmed that the monochromatic source and dichromatic source appeared to be equivalent. If all of these stopping criteria were met, then the adjustment was considered complete and the red and green settings determined during the behavioral session were saved for use during the SSVEP session. If the stopping criteria were not met and single-LED calibration was just completed, then dual-LED calibration proceeded using the current red and green D/A settings. If the stopping criteria were not met and dual-LED calibration was just completed, then single-LED calibration proceeded using the current red and green D/A settings.

SSVEP session—After the behavioral session, participants completed an SSVEP session (Exp. 1B). For three of the participants, both sessions were completed on the same day. The other six participants completed the SSVEP session on a different day (within one week). During the SSVEP session, EEG was recorded from the participants as described in Methods: EEG Recording. Following setup, each participant was given the opportunity to make final adjustments to the settings of the stimulator. After this, participants completed three runs of stimulation.

Each run of stimulation consisted of 54 six-second trials with an interstimulus interval (ISI) of one second. Previous research has shown that canonical correlation analysis (CCA) can classify SSVEPs with >75% accuracy in 2.25 s (36). The goal was to estimate the size of SSVEPs, which it was thought may require more data than classification. Thus, it was chosen to make each trial 6 s. The onset of each of these trials was detected using the digital input line of the g.USB amplifier. The order of the trials was the same in every run for every participant. Randomizing the order of the trials could have resulted in large relative luminance changes from trial to trial (see FIG. 8). Large changes in luminance could lead to large changes in pupil dilation (37), potentially introducing noise into the experiments. The first two trials of each run measured baseline levels of EEG activity. In the first baseline trial, participants were asked to attend to the stimulator while it was off. In the second baseline trial, the monochromatic source was turned on for the entire trial (i.e., there was no flicker). During each of the remaining 52 trials, the stimulator flickered at 10 Hz. This flicker was obtained using square wave stimulation (with a 50% duty cycle) that switched between the monochromatic source and the dichromatic source. The settings of the dichromatic source were fixed throughout the SSVEP session. The luminance of the monochromatic source, however, was increased from a D/A setting of 0 in the third trial by 20 D/A units each trial to 1020 D/A units in the last trial. Each run of stimulation lasted for less than seven minutes. Participants were allowed to rest for approximately five minutes between runs. The entire SSVEP session lasted less than one hour.

Experiment Two—There were two key differences between Experiment One and Experiment Two. First, during Experiment Two, the SSVEP session came before the behavioral session. Second, during the SSVEP session of Experiment Two, only the monochromatic source was fixed. The method used during the behavioral session of Experiment Two (Exp. 2A) was identical to Experiment One.

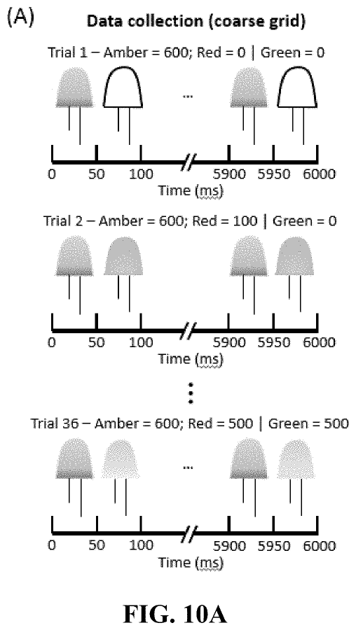

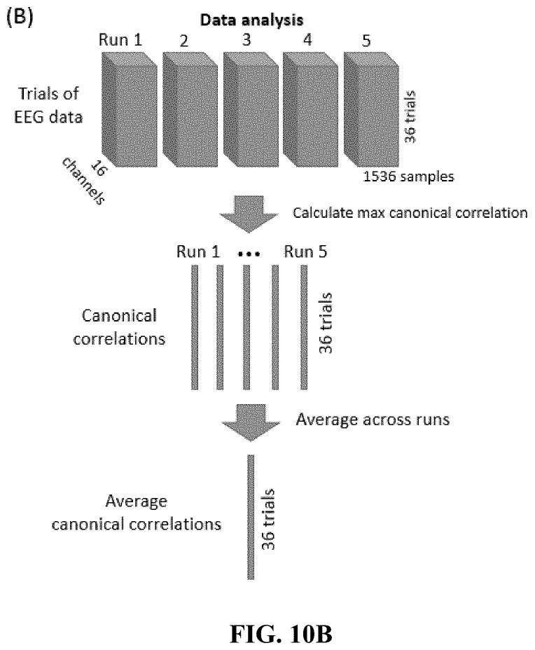

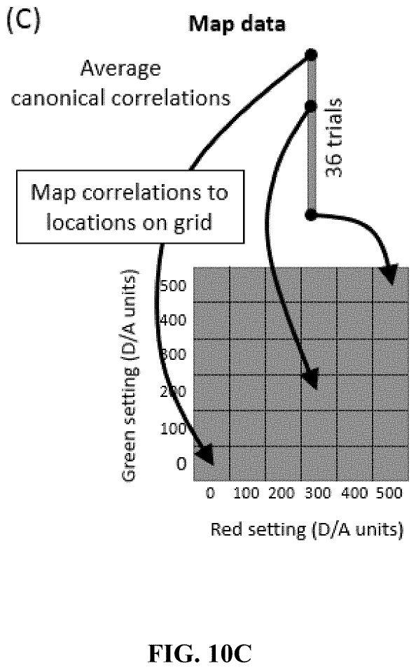

SSVEP Session (grid search)—In Experiment Two: SSVEP Session (grid search; Exp. 2B), the D/A settings of the red light and the green light that each person perceived as being metameric with the monochromatic source were assumed to be unknown. Therefore, the settings of the red light and green light that minimized the SSVEP were determined using two grid searches FIGS. 10A-10C), a coarse-grid search followed by a fine-grid search. In both searches, 36 possible combinations of red and green light settings were tested. In the coarse-grid search, green light settings of 0 to 500 D/A units in increments of 100 D/A units and red light settings of 0 to 500 D/A units in increments of 100 D/A units were tested. In the fine grid search, red light settings of 75 to 200 D/A units in increments of 25 D/A units and green light settings of 25 to 75 D/A units in increments of 10 D/A units were tested. These combinations were tested in order of increasing D/A setting (i.e., the trials were ordered from the lowest sum of the D/A settings to the highest). The D/A settings tested during the fine-grid search were chosen based on the results of the behavioral session of Experiment One.

The length of each trial, ISI, and EEG recording parameters of Experiment Two: SSVEP Session were identical to those used in Experiment One: SSVEP Session.

Experiment Three—The procedure for Experiment Three was identical to that of Experiment Two: SSVEP Session, except that the participants did not complete the fine-grid search.

Experiment Four—Experiment Four used the same EEG and Data Analysis settings as the other experiments. As opposed to conducting a grid search, however, the setting that minimized the SSVEP was identified using gradient descent based on finite differences. The stimulator was initialized to three settings chosen by the experimenter. For S401, these settings were 150 green and 150 red; 15 green and 30 red; and 0 green and 100 red. For S402, these settings were 0 green and 0 red; 0 green and 100 red; and 100 green and 0 red. For S403, these settings were 40 green and 50 red; 30 green and 100 red; and 100 green and 40 red. The system then sampled the settings surrounding the initial setting, computed a gradient, and updated its current estimate of the settings that minimized the SSVEP. This process was then repeated and continued until the difference between the different settings surrounding the current estimate was sufficiently small (i.e., a stopping criterion was met).

Data Analysis—Data analysis for all four experiments was performed using Matlab. The raw EEG data from each participant was zero-phase bandpass filtered using a 4th order IIR Butterworth filter between 3 and 45 Hz. The data was also notch filtered at 60 Hz to remove powerline noise. After filtering, individual six second trials were extracted from the EEG data. Each trial was analyzed using canonical correlation analysis (CCA) (36). CCA is a technique—widely used in BCI—for detecting SSVEPs. Here CCA is used as a relative measure of the size of the SSVEP elicited during each trial (assuming that the noise in the EEG is stable across trials). Details on the use of CCA for detecting SSVEPs was based on those described by Norton et al. (38). The CCA analysis included all 16 channels of EEG data and reference variables (sine and cosine waves) at the first, second, third, fourth, and fifth harmonic frequencies (i.e., 10, 20, 30, 40, and 50 Hz) of the stimulation. Although CCA calculates multiple canonical correlations (the number of canonical correlates is the lesser of the number of reference variables and the number of EEG channels), all but the maximum canonical correlation was discarded in the analysis. After performing CCA on each of the trials, the data from each run were normalized between zero and one.

### Example 2—Results

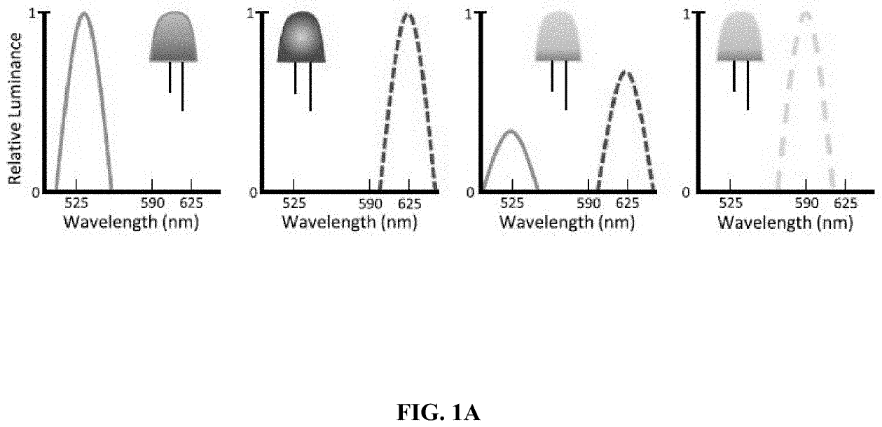

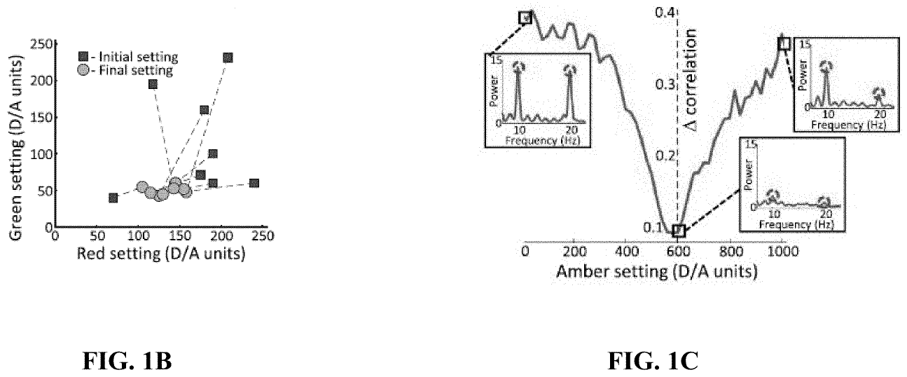

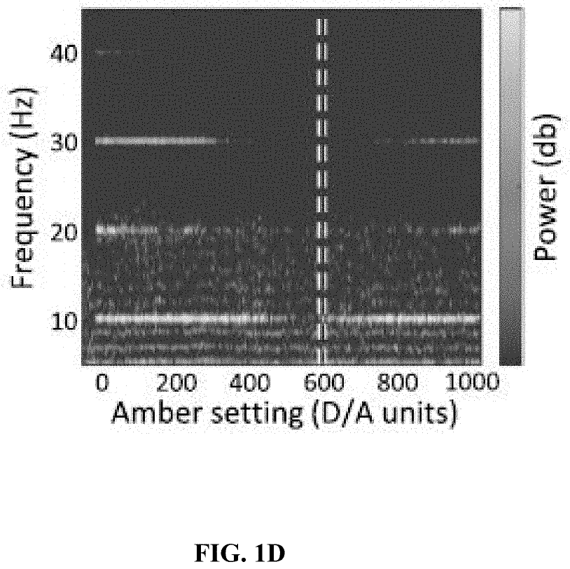

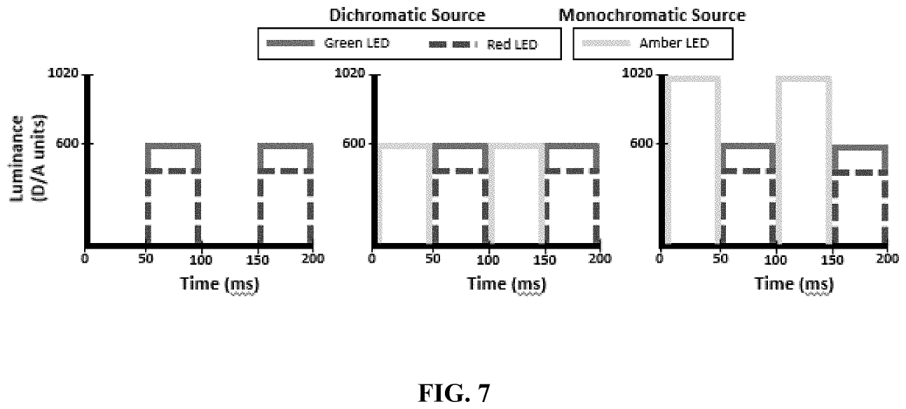

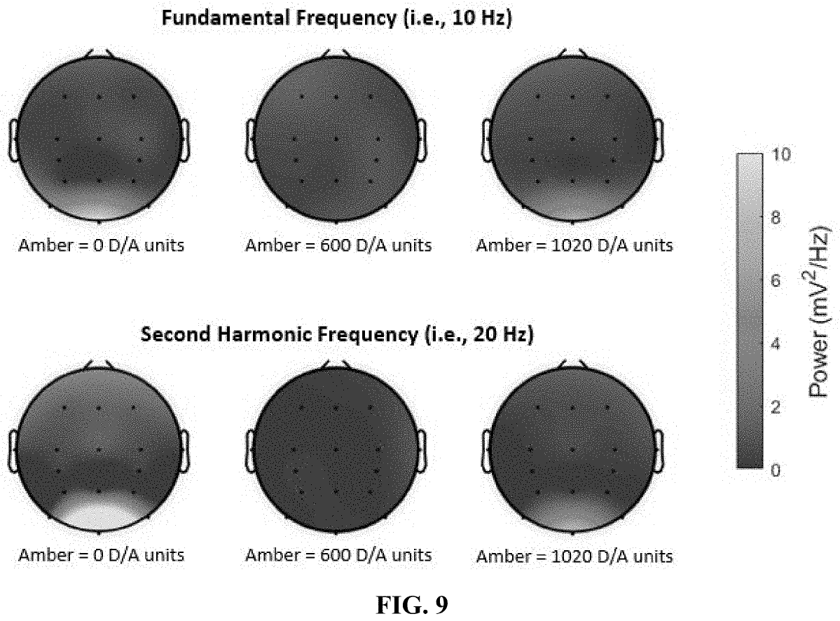

In Experiment One, behaviorally-identified metamers (Exp. 1A) were compared with those identified using SSVEPs (Exp. 1B). Participants (nine total participants, see Methods) were first asked to identify metamers by manually adjusting the dichromatic source of the stimulator to produce a color that matched the monochromatic light source, which had a fixed luminance setting of 600 digital-to-analog (D/A) units (see Methods; FIGS. 6A-6C). This process established each person' s metameric pair. The mean±SD luminance settings of the red and green components of the dichromatic source that this standard behavioral method found to be metameric with the monochromatic source were 135±18 and 51±6 D/A units, respectively (FIG. 1B). In Exp. 1B, EEG was recorded from the scalp and elicited SSVEPs by 10-Hz alternation between the two light sources. For each individual, the settings of the dichromatic source were fixed to be equal to those that Exp. 1A had found to be metamers and then adjusted the luminance of the monochromatic source from 0 to 1020 D/A units in steps of 20 (FIGS. 7 and 8; see Methods). Across eight participants (data from one person were excluded, see Methods), SSVEP size was minimized when the monochromatic source had a setting of 575±35 D/A units (FIGS. 1C and 1D; topographical plots provided in FIG. 9 (5)). This did not differ significantly from the setting of the monochromatic source found in the behavioral approach to metamer identification (i.e., 600 D/A units; one-sample Wilcoxon test, n=8, p=0.12). The SSVEPs elicited near this setting (540-720 D/A units) were significantly smaller than SSVEPs elicited at any other monochromatic source settings (one-way ANOVA; bins=0-160, 180-340, 360-520, 540-700, 720-880, and 900-1020 D/A units; p<0.001; Tukey post-hoc tests). Thus, the monochromatic source that was metameric with the dichromatic source in standard behavioral testing was also the source that minimized SSVEP amplitude.

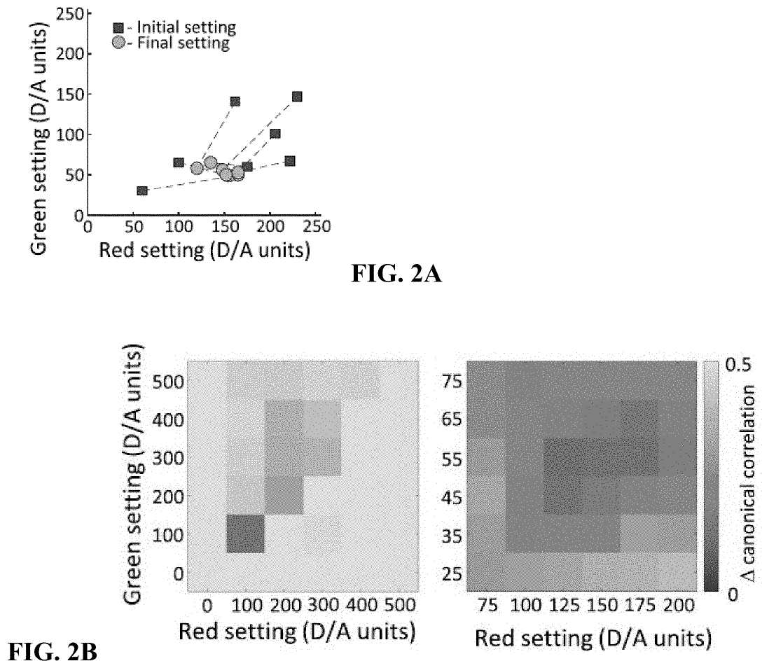

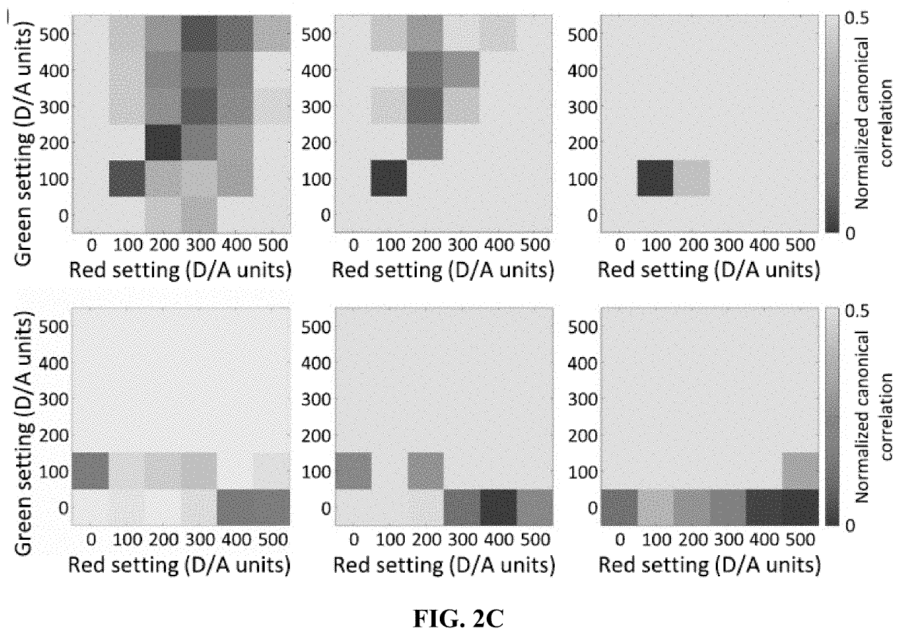

In Experiment Two (nine participants, see Methods), a more extensive search of the workspace was used to ensure that the results of Exp. 1B were not merely a local minimum. With a two-dimensional grid search, the red and green LED settings were determined (i.e., the two dimensions) of the dichromatic source that minimized the SSVEP (Exp. 2B), and then compared them with the red and green LED settings that the person identified behaviorally (i.e., manually; Exp. 2A; FIG. 2A) as metamers of the same monochromatic source used in Experiment One. For each individual, a coarse-grid search (stimulator settings of 0 to 500 D/A units in increments of 100 D/A units) was performed and then a fine grid search (red light settings of 75 to 200 D/A units in increments of 25 D/A units and green light settings of 25 to 75 D/A units in increments of 10 D/A units). In the coarse-grid search (data averaged across seven participants, see Methods), the SSVEP was minimized at 100 D/A units for both the red and green LEDs (FIG. 2B (left); analysis of grid data described in FIGS. 10A-10C). These were the closest possible settings to the behaviorally-identified metamers. In the more focused fine-grid search (averaged data from all participants is shown in FIG. 2B (right)), the SSVEP was minimized when the green LED was 56±6 D/A units and the red LED was 154±22 D/A units. These green and red settings were very close to those of the behaviorally identified metamers (54±6 for the green LED and 149±16 D/A units for the red LED) (p=0.50 and 0.75, respectively, by Wilcoxon signed-rank test. Data from three individual participants are shown in FIG. 2C (top). Thus, the more complete sampling of the workspace in Exp. 2B confirmed the SSVEP results of Exp. 1B. In summary, the data of Experiments One and Two show that the SSVEP is minimized when the two stimuli are metameric; the SSVEP can identify metamers. The SSVEP-based method described here could be improved by methodological refinements in light sources, stimulation frequency and luminance, surrounding visual conditions, and EEG signal processing. Such improvements might further reduce the minimal SSVEP produced by the metameric pair (e.g., FIG. 1C).

In Experiment Three, it was investigated whether people with CVDs could be identified using the SSVEP-based method for finding metamers. People with CVDs see colors differently. Thus, their metamers were predicted, as identified by SSVEP, would differ clearly from those of people without CVDs. To test this prediction three people with CVDs (all male) were studied. All three were identified (by the Farnsworth Dichotomous D-15 arrangement test; see (6)) as protans (i.e., they had a defective or missing L-cone). SSVEPs were elicited from these participants using the coarse-grid search of Exp. 2B (see Methods). Their results, shown in FIG. 2C (bottom), were markedly different from people without a CVD (FIG. 2C (top)) studied in Experiment Two. If these initial results are confirmed by studies in additional people, this new method could facilitate clinical detection of CVDs.

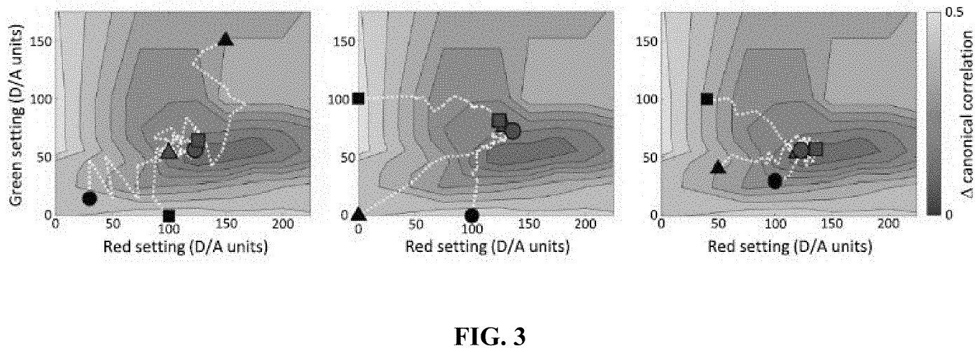

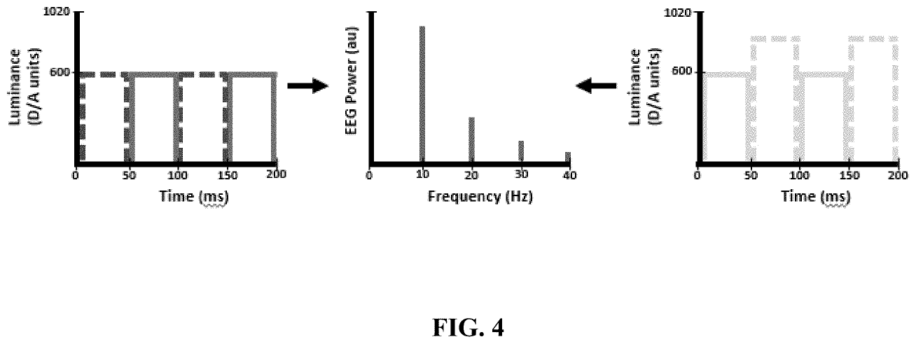

The SSVEP-based grid search is readily amenable to automated closed-loop operation. Thus, in Experiment Four, a prototype SSVEP-based BCI was tested that automatically identifies metamers. All of the tests conducted during Experiment Four were completed online (i.e., closed loop). As described in Methods, this system elicits and analyzes the person's SSVEP in real-time while iteratively adjusting the luminances of the green and red LEDs that produce the dichromatic source until the SSVEP is minimized, thereby identifying a metamer to the monochromatic amber source (the luminance of which is fixed throughout). This process of identifying the specific settings of the dichromatic source that are metameric to the monochromatic source is a two-dimensional optimization problem, which was solved using gradient descent based on finite differences. Examples of the iterative changes made by the automated system are shown in FIG. 3.

Starting from three different initial settings, the automated BCI-based system was tested in three Experiment One participants (i.e., three individuals without CVDs; see Methods). Averaged across these three runs, the automated system identified 58 green, 117 red (S401); 76 green, 130 red (S402); and 56 green, 126 red (S403) as the settings that minimized the SSVEP (i.e., were metamers to the monochromatic source) for these three individuals. As illustrated in FIG. 3, each person's three different runs arrived at very similar stimulator settings, despite starting from very different initial settings. Furthermore, a person's metameric pair identified with the automated BCI-based system was very similar to that identified behaviorally in Experiment One, and by SSVEPs in Experiment Two.

### Example 3—Discussion

In summary, Experiments One and Two show that, in people without CVDs, an SSVEP-based method can identify metameric pairs that match those identified by a standard behavioral method; Experiment Three shows that this SSVEP method can differentiate between people with and without CVDs; and Experiment Four shows that the method can be fully automated. The practical value of this new method for assessing color vision depends on its requirements and capabilities compared to current methods and on its potential for further development.

At present, color vision is evaluated mainly by behavioral methods, all of which require the attention and active participation of the person. Most prominent among these is the anomaloscope, which is the only behavioral method able to diagnose both type and severity of red-green and blue-yellow color blindness (reviewed in (7, 6)). In addition to requiring the active participation of the person being examined, the anomaloscope requires extensive training of the examiner and considerable time to administer (8). In contrast, the SSVEP-based method described here does not require the person's participation (i.e., it is a passive BCI (9)); with appropriate signal analysis, it could even be used when the person's eyes are closed (10). In addition, this new method needs minimal examiner training and can be fully automated. Furthermore, initial data (i.e., Exp. 3) indicate that it can detect protan-type CVDs accurately. (The new method can probably detect other types of CVDs as well, but this must be verified empirically.) Given these advantages SSVEP-based assessment of color vision could be particularly useful for identifying CVDs in those who cannot respond behaviorally, such as young children or people with motor or cognitive deficits.

The new SSVEP-based color-vision assessment method described here is fundamentally different from previous electrophysiological studies (11, 12, 13, 14, 15). It has four unique features. First, because it is based on the identification of metamers, it is an objective method for performing color matching, which is the gold standard of color-vision assessment. Second, by using new BCI-based procedures to analyze signals from multiple channels and frequencies, it enhances SSVEP detection and measurement (16). Third, because it poses metamer identification as an optimization problem, this new method can be fully automated and can take advantage of a wide range of powerful optimization algorithms. Fourth, the new method is generalizable; it can identify a metamer to a light source having essentially any spectral distribution.

The last two unique features—automatization and generalization—give the new method wide applicability for at least four important purposes. First, this new method could enable clinical detection and analysis of color vision deficits in young children and in others unable to participate in standard assessment methods. With further development, the method might be incorporated into a simple device that could be part of a standard pediatric examination. Second, the new method could find significant industrial applications in selecting color schemes for products and designing formats for digital displays (see (17)).

Third, because SSVEPs reflect neural activity in cortical and subcortical areas involved in visual function (18, 19), SSVEP-based detection of metamers could help to explore neural mechanisms underlying color vision (20, 21, 22). For example, it might make it possible to isolate and characterize the cortical responses to activation of intrinsically photosensitive retinal ganglion cells (ipRGCs), a photoreceptor type that contributes to visual function (reviewed in (23)).

Fourth, this new method might form the basis for the first therapeutic intervention for people with CVDs. It is now clear that the simplest reflexes are plastic; they can be changed by operant conditioning, and these changes can help to restore useful function to people with spinal cord injury (24). Given that color vision displays comparable plasticity (25), an SSVEP-based operant conditioning protocol might prove able to improve color vision in people with CVDs.

Further studies should confirm the diagnostic ability of SSVEP-based BCIs to identify the type and severity of CVDs. As a part of these studies, improvements should be made to the stimulation system (e.g., optics, calibration) and protocol (e.g., identification of the optimal number and location of EEG electrodes, monitoring of the pupil size). Lastly, these studies should include direct comparisons to existing color vision assessment methods (e.g., the anomaloscope).

In conclusion, this study describes, demonstrates, and validates a novel method for assessing color vision founded on the hypothesis that flickering visual stimuli that alternate between two metamers will not elicit an SSVEP. Unlike standard color vision assessment methods, this SSVEP-based method does not need the active participation of the person being tested. The new method provides results comparable to those of standard methods, can identify those with color vision deficits (CVDs), can be fully automated, and can be applied to a wide variety of light sources. In addition to its clear clinical diagnostic applications, SSVEP-based testing of color vision should have industrial, scientific, and possibly therapeutic applications.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

List of References cited herein is as follows, all of which are hereby incorporated by reference in their entirety:
[1] Walter T. Delpero, Hugh O'Neill, Evanne Casson, and Jeff Hovis. Aviation relevant epidemiology of color vision deficiency. Aviation, Space, and Environmental Medicine, 76(2):127-133, 2005.
[2] Theresa J. Squire, Marisa Rodriguez-Carmona, Anthony D. B. Evans, and John L. Barbur. Color vision tests for aviation: Comparison of the anomaloscope and three lantern types. Aviation, Space, and Environmental Medicine, 76(5):421-429, 2005.
[3] Michael A. Webster. Individual differences in color vision. In Andrew J. Elliot, Mark D. Fairchild, and Anna Franklin, editors, Handbook of Color Psychology, pages 197-215. Cambridge University Press, Cambridge, 2015. doi: 10.1017/cbo9781107337930.010.
[4] Francois-Benoit Vialatte, Monique Maurice, Justin Dauwels, and Andrzej Cichocki. Steady-state visually evoked potentials: Focus on essential paradigms and future perspectives. Progress in Neurobiology, 90(4):418-438, April 2010. doi: 10.1016/j.pneurobio.2009.11.005.
[5] Arnaud Delorme and Scott Makeig. EEGLAB: An open source toolbox for analysis of single-trial EEG dynamics including independent component analysis. Journal of Neuroscience Methods, 134(1):9-21, 2004.
[6] A. Fanlo Zarazaga, J. Gutierrez V'asquez, and V. Pueyo Royo. Review of the main colour vision clinical assessment tests. Archivos de la Sociedad Espanola de Ofialmolog'ia (English Edition), 94(1):25-32, January 2019. doi: 10.1016/j.oftale.2018.08.010.
[7] Stephen J. Dain. Clinical colour vision tests. Clinical and Experimental Optometry, 87(4-5):276-293, July 2004. doi: 10.1111/j.1444-0938.2004.tb05057.
[8] David V. Walsh, James Robinson, Gina M. Jurek, Jose E. Capo-Aponte, Daniel W. Riggs, and Leonard A. Temme. A performance comparison of color vision tests for military screening. Aerospace Medicine and Human Performance, 87(4):382-387, 2016.
[9] P Arico, G Borghini, G Di Flumeri, N Sciaraffa, and F Babiloni. Passive BCI beyond the lab: Current trends and future directions. Physiological Measurement, 39(8):08TR02, 2018.
[10] James J. S. Norton, Stephen Umunna, and Timothy Bretl. The elicitation of steady-state visual evoked potentials during sleep. Psychophysiology, 54 (4):496-507, 2017.
[11] Dale Allen, Martin S. Banks, and Anthony M. Norcia. Does chromatic sensitivity develop more slowly than luminance sensitivity? Vision Research, 33:2553-2553, 1993.
[12] Jurgen Gerling, Thomas Meigen, and Michael Bach. Diagnosis of protan and deutan color vision deficiencies with pattern-ERG and VEP. In Colour Vision Deficiencies XII, pages 375-380. Springer, 1995.
[13] James N. Ver Hoeve, Thomas D. France, and G. Andrew Bousch. A sweep VEP test for color vision deficits in infants and young children. Journal of Pediatric Ophthalmology and Strabismus, 33(6):298-302, 1996.
[14] Bruno D. Gomes, Givago S. Souza, Anderson R. Rodrigues, Cezar A. Saito, Luiz Carlos L. Silveira, and Manoel Da Silva Filho. Normal and dichromatic color discrimination measured with transient visual evoked potential. Visual Neuroscience, 23(3-4):617-627, 2006.
[15] Jeff C. Rabin, Andrew C. Kryder, and Dan Lam. Diagnosis of normal and abnormal color vision with cone-specific veps. Translational Vision Science & Technology, 5(3):8-8, 2016.
[16] Wenya Nan, Chi Man Wong, Boyu Wang, Feng Wan, Peng Un Mak, Pui In Mak, and Mang I. Vai. A comparison of minimum energy combination and canonical correlation analysis for SSVEP detection. In 2011 5th International IEEE/EMBS Conference on Neural Engineering, pages 469-472. IEEE, 2011.
[17] R. Hunt. The reproduction of colour sixth edition. John Wiley & Sons, 2004.
[18]Francesco Di Russo, Sabrina Pitzalis, Teresa Aprile, Grazia Spitoni, Fabiana Patria, Alessandra Stella, Donatella Spinelli, and Steven A. Hillyard. Spatiotemporal analysis of the cortical sources of the steady-state visual evoked potential. Human Brain Mapping, 28(4):323-334, 2007. doi: 10.1002/hbm.20276.
[19] Mana A. Pastor, M. Valencia, Julio Artieda, Manuel Alegre, and Jose C. Masdeu. Topography of cortical activation differs for fundamental and harmonic frequencies of the steady-state visual-evoked responses. An EEG and PET H₂¹⁵O study. Cerebral Cortex, 17(8):1899-1905, October 2007. doi: 10.1093/cercor/bh1098.
[20] Russell L. De Valois and Karen K. De Valois. A multi-stage color model. Vision Research, 33(8):1053-1065, 1993.
[21] Katherine Mancuso, Matthew C. Mauck, James A. Kuchenbecker, Maureen Neitz, and Jay Neitz. A multi-stage color model revisited: Implications for a gene therapy cure for red-green colorblindness. In Retinal Degenerative Diseases, pages 631-638. Springer, New York, 2010.
[22] Brian P. Schmidt, Maureen Neitz, and Jay Neitz. Neurobiological hypothesis of color appearance and hue perception. Journal of the Optical Society of America A, 31(4):A195-A207, 2014.
[23] Tiffany M. Schmidt, Shih-Kuo Chen, and Samer Hattar. Intrinsically photosensitive retinal ganglion cells: Many subtypes, diverse functions. Trends in Neurosciences, 34(11):572-580, 2011.
[24] James J. S. Norton and Jonathan R Wolpaw. Acquisition, maintenance, and therapeutic use of a simple motor skill. Current Opinion in Behavioral Sciences, 20:138-144, 2018.
[25] Jay Neitz, Joseph Carroll, Yasuki Yamauchi, Maureen Neitz, and David R. Williams. Color perception is mediated by a plastic neural mechanism that is adjustable in adults. Neuron, 35(4):783-792, August 2002. doi: 10.1016/s0896-6273(02)00818-8.
[26] Shinobu Ishihara. Tests for color blindness. American Journal of Ophthalmology, 1(5):376, May 1918. doi: 10.1016/s0002-9394(18)90663-x.
[27]Dean Farnsworth. Farnsworth dichotomous test for color blindness. 1947.
[28] G. E. Chatrian, E. Lettich, and P. L. Nelson. Ten percent electrode system for topographic studies of spontaneous and evoked EEG activities. American Journal of EEG Technology, 25(2):83-92, June 1985. doi: 10.1080/00029238.1985.11080163.
[29] G. Schalk, D.J. McFarland, T. Hinterberger, N. Birbaumer, and J. R. Wolpaw. BCI2000: A general-purpose brain-computer interface (BCI) system. IEEE Transactions on Biomedical Engineering, 51(6):1034-1043, June 2004. doi: 10.1109/tbme.2004.827072.
[30] Rolf G. Kuehni. Variability in estimation of suprathreshold small color differences. Color Research & Application, 34(5):367-374, October 2009. doi: 10.1002/col.20522.
[31] John W. T. Walsh. Modern photometry. Nature, 120(3013):157-157, July 1927. doi: 10.1038/120157a0.
[32] Richard A. Bone and John T. Landrum. Heterochromatic flicker photometry. Archives of Biochemistry and Biophysics, 430(2):137-142, October 2004. doi: 10.1016/j.abb.2004.04.003.
[33] Petteri Teikari, Raymond P Najjar, Kenneth Knoblauch, Dominique Dumortier, Pierre-Low Cornut, Philippe Denis, Howard M Cooper, and Claude Gronfier. Refined flicker photometry technique to measure ocular lens density. JOSA A, 29(11):2469-2478, 2012.
[34] Shao-Min Hung, Dan Milea, Annadata Venkata Rukmini, Raymond P. Najjar, Joo Huang Tan, Fran ̧soise Vienot, Marie Dubail, Sharon Lee Choon Tow, Tin Aung, Joshua J. Gooley, and Po-Jang Hsieh. Cerebral neural correlates of differential melanopic photic stimulation in humans. NeuroImage, 146:763-769, February 2017. doi: 10.1016/j.neuroimage.2016.09.061.
[35] Timothy M. Brown, Seiichi Tsujimura, Annette E. Allen, Jonathan Wynne, Robert Bedford, Graham Vickery, Anthony Vugler, and Robert J. Lucas. Melanopsin-based brightness discrimination in mice and humans. Current Biology, 22(12):1134-1141, June 2012. doi: 10.1016/j.cub.2012.04. 039.
[36] Zhonglin Lin, Changshui Zhang, Wei Wu, and Xiaorong Gao. Frequency recognition based on canonical correlation analysis for SSVEP-based BCIs. IEEE Transactions on Biomedical Engineering, 54(6):1172-1176, June 2007. doi: 10.1109/tbme.2006.889197.
[37] Andrew B Watson and John I Yellott. A unified formula for light-adapted pupil size. Journal of Vision, 12(10):12-12, 2012.
[38] James J. S. Norton, Jessica Mullins, Birgit E. Alitz, and Timothy Bretl. The performance of 9-11-year-old children using an SSVEP-based BCI for target selection. Journal of Neural Engineering, 15(5):056012, July 2018. doi: 10.1088/1741-2552/aacfdd.

1. A method for automated assessment of color vision, said method comprising:
identifying one or more steady-state visual evoked potentials (SSVEPs) under conditions effective to automatically assess color vision.

2. The method of claim 1, wherein color vision is assessed by identifying metamers.

3. The method of claim 2, wherein said metamers are produced by at least two light sources.

4. The method of claim 3, wherein said at least two light sources comprise a visible light source.

5. (canceled)

6. The method of claim 3, wherein said at least two light sources are alternating.

7. The method of claim 3, wherein said at least two light sources differ in saturation, color, hue, luminance, or any combination thereof.

8. The method of claim 1 further comprising:
providing a subject having or suspected of having a color vision deficiency.

9. The method of claim 8 further comprising:
comparing presence of a response to at least two light sources in said subject having or suspected of having a color vision deficiency to a control response to said at least two light sources.

10. The method of claim 9, wherein, when said response to said at least two light sources is within a specific range, said response indicates that said subject has or is likely to have a color vision deficiency.

11. A device for automated assessment of color vision, wherein said device compares steady-state visual evoked potentials (SSVEPs) elicited by at least two light sources comprising different spectral distributions.

12. The device of claim 11 further comprising:
a brain computer interface (BCI) to compare said SSVEPs.

13. The device of claim 12, wherein said BCI uses an electroencephalogram (EEG) or other neural imaging system measurement.

14. The device of claim 11 further comprising:
providing alternating at least two light sources.

15. The device of claim 14, wherein said at least two light sources differ in saturation, color, hue, luminance, or any combination thereof.

16.-19. (canceled)

20. A system for automated assessment of color vision, said system comprising:
providing at least two alternating light sources, and
measuring one or more steady-state visual evoked potentials (SSVEP) elicited by said at least two light sources.

21. The system of claim 20, wherein said system is used to visualize information processing in one or more visual pathways of a subject.

22. The system of claim 20, wherein a brain computer interface (BCI) is used to assess color vision.

23. The system of claim 22, wherein said system identifies one or more metamers.

24. The system of claim 20, wherein said at least two light sources comprise a visible light source.

25. (canceled)

26. The system of claim 21, wherein said subject has or is suspected of having a color vision deficiency.

27. The system of claim 26, wherein the SSVEPs in said subject having or suspected of having a color vision deficiency is compared to SSVEPs in a control sample.

28. The system of claim 27, wherein, when said SSVEPs in said subject having or suspected of having a color vision deficiency is within a specific range, said SSVEPs indicate that said subject has or is likely to have a color vision deficiency.

29. The system of claim 20, wherein said system provides real-time analysis of brain activity.

30. The system of claim 20, wherein a relative color of two light sources is adjusted until one or more metamers are identified.

31. A method of modifying color vision, said method comprising:
measuring one or more steady-state visual evoked potentials (SSVEPs) elicited in response to at least two light sources, and
providing feedback on SSVEP measurements to a subject under conditions effective to modify color vision.

32.-36. (canceled)

37. The method of claim 1, wherein a brain computer interface (BCI) is used to assess color vision.

38. The method of claim 1, wherein assessment of color vision is carried out after SSVEPs are identified.

39. The method of claim 1, wherein assessment of color vision is carried out simultaneously with identification of SSVEPs.

40. The method of claim 8 further comprising:
comparing presence of SSVEPs in said subject having or suspected of having a color vision deficiency to SSVEPs elicited in a subject with no color vision deficiency.

41. The method of claim 40, wherein, when said SSVEPs are within a specific range, said SSVEPs indicates that said subject has or is likely to have a color vision deficiency.

42. The device of claim 11, wherein said alternating light sources are metamers.

43. The method of claim 31 further comprising:
treating color vision deficiency in said subject.

44. The method of claim 31, wherein said at least two light sources differ in saturation, color, hue, luminance, or any combination thereof.

45. The method of claim 43, wherein said treating comprises:
measuring SSVEPs in response to said at least two light sources, and
administering a treatment for a color vision deficit.

46. The method of claim 31, wherein said feedback is used to teach the subject to modify SSVEPs.

47. The method of claim 46, wherein the modified SSVEPs are measured to determine improvement in color vision in said subject.

48. The method of claim 31, wherein said feedback is in a format selected from the group consisting of visual, tactile, auditory, or any combination thereof.
